(19) United States
(12) Patent Application Publication
(10) Pub. No.: US 2025/0364113 A1
(43) Pub. Date: Nov. 27, 2025

(54) HUMAN-COMPUTER INTERFACES FOR MUSICAL INSTRUMENTS

(71) Applicant: Yehoyariv SOREK, Netanya (IL)

(72) Inventor: Yehoyariv SOREK, Netanya (IL)

(21) Appl. No.: 19/215,919

(22) Filed: May 22, 2025

Related U.S. Application Data
(60) Provisional application No. 63/650,575, filed on May 22, 2024.

(51) Int. Cl. G16H 20/70; G10H 1/00

(52) U.S. Cl. CPC G16H 20/70; G10H 1/0008; G10H 2220/201; G10H 2240/175

(57) ABSTRACT

A system for music, physical therapy and rehabilitation has been developed that allows for differently-abled users of many types to create and perform music by use of their bodies, in ways found to be physiologically therapeutic. Many use cases including rehabilitation, physical therapies of various types, music therapies of different types and especially the combination of music and physical therapies are made possible by the system which uses a set of human-computer-interaction devices for input, such as eye trackers, motion trackers, IMUs and so on as input devices to a computer system outfitted with a number of software applications. The software applications in turn control outputs including music production systems as well as lighting and in some cases physical instruments, and the user can choose from a wealth of options to create a self-guided program of improvement.

This application claims the benefit of priority of U.S. Provisional Patent Application No. 63/650,575, filed May 22, 2024, the contents of which are all incorporated herein by reference in their entirety.

### BACKGROUND OF THE INVENTION

### Background of the Invention

The history of human culture is intertwined with the development of music, to the extent that musical instruments are found amongst the earliest human artifacts known. The practical aspects of musical enterprises have developed since the introduction of computers, with composition, production, performance and dissemination all coming into the digital realm.

Likewise, the use of computers in physical and occupational therapies for people of limited mobility has seen tremendous progress, with computers being used for creating therapeutic programs, tracking progress, and facilitating measurements of various sorts.

U.S. Pat. No. 8,111,239B2 “Man machine interfaces and applications” provides affordable methods for input of position, attitude (orientation) and other data to computers for the purpose of CAD, painting, aids to the disabled, and internet or other experiences. Electro-optical sensors and particularly TV cameras are used to provide data from external or natural features on objects. However this device is limited to provision of position data and neither does it deal with users of limited mobility.

KR 20180067060A “Music Therapy” provides a method of music therapy for behavioral and mental development and treatment of growing children, adapted to develop communication abilities of children, improve motor coordination, and cultivate social awareness and creativity. However the application does not provide for interaction with users of limited mobility.

U.S. Pat. No. 10,437,335B2 “Wearable electronic, multi-sensory, human/machine, human/human interfaces” provides for a wearable Haptic Human/Machine Interface (HHMI) which measures electrical activity from muscles and nerves of a user, which are then amplified and processed. Exemplary uses include mitigating tremor, accelerated learning, cognitive therapy, remote robotic, drone and probe control and sensing, virtual and augmented reality, stroke, brain and spinal cord rehabilitation, education, pain relief, remote surgery, biofeedback and so on. However while the application may possibly provide for interaction with users of limited mobility, there is no explicit provision for rehabilitation

CN201346346Y “Flute-based therapeutic device” provides a flute-based therapeutic device having a flute head, a playing tube, a sound-generating tube and medical-use magnets. The device is intended to promote the mental and physical health of its users. However while the application does provide for computer-mediated musical expression, there is no explicit provision interaction with users of limited mobility nor for rehabilitation.

### SUMMARY OF THE INVENTION

Technological advancements have significantly transformed the way we communicate, work, and interact with the world around us. However, many individuals with limited mobility, such as those with motor impairments, find it challenging to access and engage with computer systems effectively. Traditional input devices, such as keyboards and mice, often pose significant barriers for these individuals, limiting their ability to interact with computers and digital interfaces independently.

Recognizing the need to empower individuals with limited mobility, the present invention introduces a comprehensive system and method that revolutionizes HCl through the incorporation of novel computer instruments. This system aims to bridge the accessibility gap, allowing individuals with limited mobility to seamlessly navigate and interact with various software applications, websites, and digital content.

The primary objective of this patent is to provide a set of inclusive HCl solutions that accommodates individuals with diverse physical impairments and offers them an equal opportunity to learn and enjoy music as well as benefit from the increased mobility, range of motion and so on provided by targeted physical and occupational therapies combined with music. By harnessing the power of emerging technologies, the proposed system enables users to overcome mobility limitations and enjoy an enhanced, intuitive, and personalized user experience.

As will be clear to those skilled in the art, occupational therapy involves manual and fine motor skills, and specific basic life tasks such as eating, drinking, toilet functions, dressing, and bathing. Furthermore there are emotional and cognitive aspects to occupational therapy, for example involving memory, an aspect specifically targeted by the invention in several of its embodiments.

Physiotherapy, on the other hand, is more concerned with range of motion, walking, transitions, and gross motor function. The invention provides for these as described herein, generally by means of encouraging increased range of motion by allowing the user to control instruments at the ‘edges’ of their ranges of motion, such that slight range-of-motion improvements are necessary in order to fully play the various instruments.

Emotional therapy is also a part of the invention; by allowing users to express themselves with music, the users find satisfaction and enjoyment important to their emotional state and development. The notions of music therapy and music-as-therapy are applied here to a great degree. This occurs for instance by means of playing instruments; the music often serves as a trigger for improvement. The treatment itself happens through the music, for example playing music is often a trigger for therapeutic progress. As another example. hearing a song with emotional content may often lead to talking and going over emotional points with a therapist. There is an overlap between the approaches of emotional therapy and music therapy and between music therapy and making or hearing music. By use of the inventive system, it is possible to make significant emotional advances in an indirect way. It is possible to reach significant emotional therapeutic results for patients, through the contents of the system and through the profession of music therapy.

In the following sections, we will delve into the key features, components, and functionality of the novel HCl computer instruments. We will discuss how the system integrates seamlessly with existing software and hardware platforms, fostering a more accessible and inclusive digital environment for individuals with limited mobility. Furthermore, we will explore the potential applications, benefits, and anticipated impact of this innovative system on the lives of its users.

The introduction of these novel HCl computer instruments holds great promise in revolutionizing the way many individuals engage with computers and digital interfaces. Groups specifically benefiting from the invention include those having limited mobility, limited cognitive function, seniors, and so on, but many of the inventive provisions can be used to good effect by any user. By empowering users with an intuitive and accessible platform, the invention aims to enhance their independence, productivity, and overall quality of life. The methods allow not only for increased ranges of motion, but are a way to improve the user's ability to recover from various deficits.

The invention provides a method for facilitating paramedical rehabilitation towards a therapeutic goal for a user comprising steps of:
a. providing a computer with HCl hardware adapted for gathering input from said user;
b. gathering input from said user to define a set of gestures;
c. using said gestures to control aspects of music production by software running on said computer;
d. requiring said user to broaden the range of motion required to produce said gestures, as determined by a paramedical therapist;
whereby to which the production of music is controlled by the user performing gestures which in turn serve to progress towards said therapeutic goal.

The invention further provides a method adapted to enable progress in rehabilitative processes and treatments through creating and playing music using a variety of HCl input methods to allow users of various abilities to interact with a music creation system.

The invention comprises a system for music creation and physical rehabilitation comprising:
a. one or more HCl input devices including gesture, eye-tracking, and voice input modules;
b. a processor configured to translate said input into real-time musical output;
c. a calibration module that allows automatic input mapping and user definitions for precise personalization according to patient needs;
d. a feedback module providing visual and auditory cues;
wherein the system enables personalized, adaptive music-based therapy.

The invention comprises storing user progress over time and visualized using interactive graphs showing improvements in range of motion, speed, static maintenance of movement, walking distance, clicks, volume of speech, accuracy, or cognitive engagement.

The invention further comprises use of facial recognition or expression detection to control therapy scenarios.

The invention further allows for musical collaboration supported among multiple users in real time via a networked environment.

The invention further comprises means for calibrating a user's limited mobility range for use in a musical interface, including means for:
a. automatically or manually detecting physical motion of a body part;
b. automatically or manually mapping said motion to a range of virtual instrument control;
c. progressively increasing target motion thresholds;
whereby therapeutic motion targets are integrated into musical performance tasks.

The invention further allows for gesture recognition including user-defined gestures captured through depth-sensing cameras and machine learning algorithms.

The invention further comprises an eye-tracking module that allows users to trigger musical notes or instrument parameters based on gaze position, duration, or movement direction.

The invention further comprises a voice input module configured to detect vocal parameters including pitch, amplitude, and phoneme clarity to control system outputs and provide speech modification and feedback.

The invention further comprises real-time monitoring of user physiological data including heart rate, respiration, and muscle tension to modulate musical parameters as biofeedback.

The invention further comprises virtual and physical instruments triggered by user movement through actuators connected to the control system.

The invention further comprises software including integration with commercial music production environments.

The invention further comprises a library of therapeutic programs tailored for distinct user populations, including children with autism, elderly users, and stroke survivors.

The invention further comprises an HCl interface allows configuration by a therapist to set treatment goals, assign motion-to-music mappings, and monitor user performance data.

The invention further comprises a drawing module enabling users to paint or sketch onscreen using gaze or motion input synchronized with music playback.

The invention further comprises customizable virtual avatars or mirror characters that reflect or amplify the user's actions to encourage emotional engagement and perceived self-efficacy.

The invention further comprises a mode for triggering predefined musical sequences upon detection of movement patterns involving multiple body parts in synchrony.

The invention further comprises a step of associating specific gestures with musical notes, chords, or rhythm patterns.

The invention further comprises mapping including nonlinear scaling between physical and virtual ranges to accommodate differential control precision across motion extents.

### BRIEF DESCRIPTION OF DRAWINGS

FIG. 1 shows a system diagram of the invention.

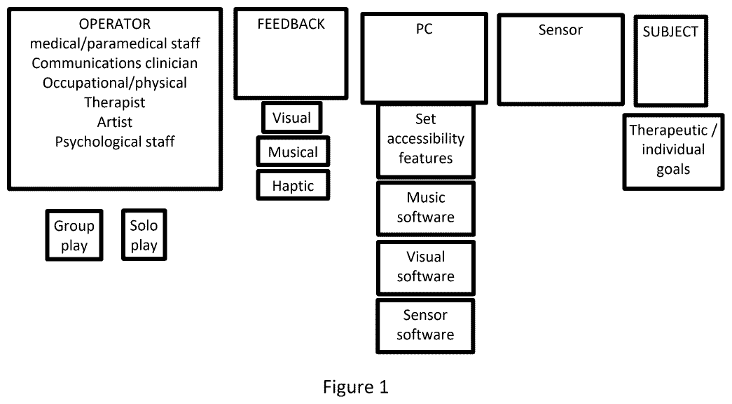

FIG. 2 shows a further system diagram of the invention.

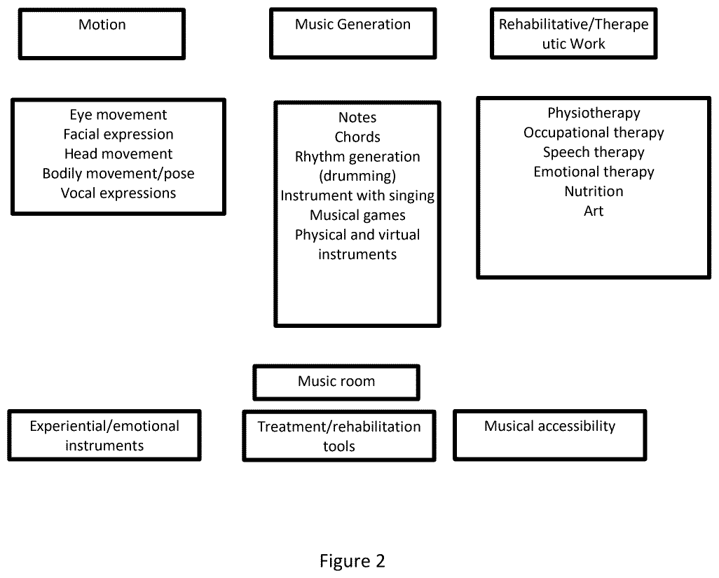

FIG. 3 shows a further system diagram of the invention.

FIG. 4 shows a graph of user response measurements over time.

FIG. 5 shows a control screen of the invention, allowing operator control over system parameters.

### DEFINITIONS

In the following detailed description, terms will be defined as follows:

“Gestures” include physical actions; movements; vocal output such as words, sounds, or utterances; or any other measurable change in the physical world that a user may cause.

“HCl” refers to any human-computer interface including but not limited to haptic interfaces, eye trackers, video cameras, depth cameras, microphones, electronic instrument interfaces, and so on.

### DETAILED DESCRIPTION OF THE INVENTION

The HCl computer instruments described in this patent offer a range of innovative solutions to enable individuals with limited mobility to interact with computers and digital interfaces effectively. These instruments incorporate advanced technologies, including gesture recognition, voice control, and eye-tracking, to provide intuitive and accessible means of input and navigation. Embedded microcontrollers such as the Arduino or similar are employed for implementation of hybrid physical/virtual instruments.

### Gesture Recognition

The gesture recognition feature of the HCl computer instruments allows users to control computer systems through natural hand and body movements. Utilizing depth-sensing cameras and/or similar sensors, the system captures and interprets a set of user-definable gestures made by the user, translating them into specific commands or actions.

By leveraging this technology, individuals with limited mobility can perform tasks such as scrolling, clicking, and navigating through menus without relying on traditional input devices. Users may define their own gestures by means of example, with the system employing for instance machine learning techniques to allow users to train the system with their own personal gestures for given purposes. This is especially useful since the range of motion and physical movement ability of different users may be radically different. Thus users with severe palsies, Parkinson's or other motor function degradation are able to define gestures suitable for their own capabilities. These gestures may then be used with the rest of the system as described below, to manipulate various musical instruments in ways that facilitate various forms of therapy (including physical therapy and speech therapy) in an enjoyable environment.

### Voice Control

Voice control functionality enables individuals with limited mobility to interact with computers and digital interfaces through speech commands. The HCl computer instruments employ sophisticated voice recognition algorithms that accurately capture and interpret spoken instructions. Users can dictate text, execute commands, launch applications, and control various software functionalities solely through their voice. This feature not only enhances accessibility but also facilitates hands-free operation, promoting convenience and efficiency. By allowing for speech-to-text in multiple languages, the system may be easily localized to allow for use in different countries and geographic regions. Voice amplitude, pitch, facial expressions as they effect voice and speech, speech clarity, and so on are all available to the system software to allow for various programs of instruction and feedback to help the patient improve. The system is able to recognize words, voices, timbre, amplitude, pitch, and other physical characteristics of the user's voice, and to use these measurements to achieve various goals of speech therapy such as audibility, coherence, understandability, improved delivery and diction, and vocabulary improvement (by means of introducing new words adapted for a given patient). Generally speaking, the use of music for speech therapy is a strong point of the invention, as it allows both for musical expression and linguistic expression in a framework allowing for measurement and feedback.

The system may combine music and speech, for example defining words and phrases that activate various multimedia of the system giving an expression of color and music. For example, a well-pronounced word will cause the system words play notes. In general, the system is structured for the ability to create personalized tools for patients with musical, visual content in movement and combination of movements. It is possible to combine, for example, a user mouse controlled by user movement, voice, and speech.

### Eye-Tracking

Eye-tracking technology plays an important role in some embodiments of the HCl computer instruments of the invention, providing an alternative means of input and control. By leveraging specialized eye-tracking hardware, such as infrared cameras, the system accurately tracks the user's eye movements and translates them into corresponding actions on the computer screen. Individuals with limited mobility can navigate through graphical user interfaces, select options, and interact with on-screen elements simply by moving their eyes. Actions such as playing musical notes on a virtual instrument may be taken either when the pointer enters/exits a particular area or when hovering; by this means users with different types of motor control can use the system without frustration.

The standard use of gaze tracking for the populations intended for the invention is far from the invention's use thereof. Gaze tracking us included in the invention directly to make music, to change parameters of the system, to allow for playing a physical or physical/virtual instrument, and to allow a bit of creativity. For example, use of eye tracking allows for simultaneous onscreen drawing and music-playing, the ability to play professionally, and so on. Furthermore the gaze tracking as used in the invention allows for modularity and customization for different patients. Gaze focus enables use at an advanced level without requiring physical contact, which allows the system to give patients tools for creating music at a relatively advanced level. The invention allows the connection of existing hardware eye trackers to the inventive system capabilities including those for creating music. The eye-tracking hardware allows for a connection between the patient's ability to focus a gaze to advanced abilities and does not emphasize the very use of eye tracking, or by using different body gestures and machine learning, etc. By use of these various means there are more possibilities for innovation when operating the system.

### Further Sensors

Further sensors may be used with the system, such as movement and IMU sensors, microphones, and so on. These may be interfaced with embedded microcontrollers such as the Arduino or similar in order to send sense data to software of the invention as described below.

Integration with Existing Software and Hardware

The HCl computer instruments are designed to seamlessly integrate with existing software applications and hardware platforms, ensuring compatibility and ease of use. Through the use and development of device drivers and software plugins, the system can be integrated with various operating systems, music composition and production suites, web browsers, and other software tools. This compatibility allows users to leverage the full functionality of their preferred applications while benefiting from the accessibility enhancements provided by the HCl instruments.

### Personalization and Customization

Recognizing the diverse needs of individuals with limited mobility, the HCl computer instruments offer extensive personalization and customization options. Users can tailor the system to their specific requirements, including adjusting input and output language, sensitivity levels, defining gesture patterns, configuring voice commands, and adapting eye-tracking parameters. This flexibility ensures that the HCl instruments can adapt to the unique needs and preferences of each user, maximizing their overall experience and usability. The individual, personalized adjustments can be set (by the therapist or patient, depending upon the situation) according to therapeutic goals for each patient and also according to the patient's cognitive and physical abilities. Different instruments can be tried and modified, to adapt to the specific cognitive abilities and needs of the user.

Other technology elements that can be used for personalization include dwell time until the music starts/stops, and mapping the eye-tracking and other controls to different system controls. For example, detection of the direction of eye movement (up/down and right/left) can be mapped to different controls such as increasing the volume (up-down) and choosing an instrument (right-left), for example piano vs. violin. The method allows for the ability to integrate with a variety of options: it gestures such as raising a hand may be mapped to control functions, or multiple gestures may be combined such as requiring both raising a hand and eye movement to effect a specific control.

### Applications and Benefits

The HCl computer instruments have a wide range of applications across various sectors. In educational settings, they empower students with limited mobility to actively participate in digital learning environments, for example music instruction classes. These instructional sessions may also be carried out remotely. Additionally, the HCl instruments facilitate social inclusion by enabling individuals with limited mobility to engage in online communication, music production, composition, and performance platforms, and virtual environments. The variety of the system's capabilities allows a variety of populations to use the system, in educational settings and in musical and experiential use. The method provides a rehabilitation tool for a wide variety of populations and not only for those with disabilities, for example people after surgeries, seniors and the elderly, children with special needs (general and not only physical disabilities, for example, speech difficulties, autism etc.) and normal children in regular educational settings.

The benefits of the HCl computer instruments extend beyond accessibility. By providing individuals with limited mobility the means to interact with computers independently, these instruments promote empowerment, boost self-esteem, and enhance overall quality of life. They foster greater productivity, allowing users to overcome physical limitations and achieve their full potential in various personal and professional endeavors.

The HCl computer instruments described in this patent offer a groundbreaking solution to enhance human-computer interaction for individuals with limited mobility. By incorporating gesture recognition, voice control, and eye-tracking technologies, this system breaks down accessibility barriers and enables individuals to interact with computers and digital interfaces effortlessly. The integration with existing software and hardware platforms, coupled with extensive personalization options, ensures adaptability and usability for users with diverse needs. The potential applications and benefits of these HCl instruments are vast, offering individuals with limited mobility newfound independence, productivity, and an enriched digital experience. On the emotional side, the feeling of success and ability to create adds to the feeling of equality and ability. For example, one can have mixed groups of both normal children and those with disabilities, who together can create musical works. Thus the use of human-to-computer interaction can be said to be not only for the disabled.

It is within provision of the invention to allow the user to control which body part controls any given musical instrument. Icons representing body parts may be selected using an initial input means such as joystick or gesture control, after which each body part can be associated with an instrument. Subsequently, the chosen body part will control the given instrument.

Thus a person who is only able to (for instance) make slight head movements can control the system in its entirety. Furthermore a user who wants to (for example) work on their left hand mobility can define the left hand as the main controller. The ranges of motion are also user-definable such that (for instance) a full range of up-down motion is controlled by head movement between two given positions of head motion up and down, and similarly a full range of left-right motion is controlled by head movement between two given positions of head motion left and right. By ‘full range’ here we mean for instance the full travel range of a mouse or other pointing device. These ranges can be manipulated by the user in a number of ways, first and foremost by means of a calibration procedure that lets the user define the actual range of physical motion or part thereof, and also the range of pointer motion to ascribe to this physical motion. It is within provision of the invention that nonlinear relations between physical motion and pointer motion be defined, allowing users with (for instance) fine control in a certain range of motion but only coarse control in another range to better control the pointer by (for example) ascribing larger ranges of pointer motion for physical ranges in which they have finer control. Within the scope of the invention, it is possible to allow the user to control which body part/voice controls the way the music is created (our addition), for example a certain body part controlling a certain aspect of a (virtual) musical instrument. The method of visual representation can be chosen for the selected trigger/body part using a primary input device such as a joystick or gesture control, and then each body part can be associated with the device. The selected body part will then control the given device.

The ranges may be defined in absolute or relative terms, and may also be defined using input devices. For instance the user may be instructed in a calibration step to perform his/her maximum range of motion. The user or another operator may then for instance set a goal that this range be extended by 10%. The subsequent operation of the system will then attempt to reach this goal, by (for instance) placing objects to reach just out of the known range of the user. In this way the system can be helpful in increasing the range of motion of the user. Likewise, precision of motion, dynamic or static equilibrium, strength, and other characteristics of motion may be set as goals for the user of the system to develop, by means of targeted exercises, games or other activities implemented by the system.

As an example of an exercise adapted for development of static equilibrium, having the user ‘hold’ virtual balls in the air (with their hands for instance outstretched or raised) in order to make or maintain a computer-implemented sound will encourage the user to keep their hand at this same level (since any other position will for instance allow the sound to decay). This will tend to reward the user's maintaining static equilibrium.

It is within provision of the invention to use timed modes that require the user to make certain motions within certain time frames, for example to keep the beat of a clip of music being played by hitting a virtual drum in time with the music. This may take the form of a game in which better-timed reactions on the part of the user are rewarded with more points, or greater progress through the game, or the like.

It is within provision of the invention to carry out an ‘intake procedure’ whereby the physical limitations and/or capabilities of a given user are input and/or learned by the system, including user preferences as to what languages are preferred for instruction, what limbs and/or ranges of motion should be ‘worked’ or utilized for control and which should be used for therapy, what types of music are preferred, what types of exercises to use, and so on.

The music production elements of the system may be based on commercial software packages such as Unity, Ableton and the like.

As mentioned it is within provision of the invention to utilize automatic range detection and mapping according to actual or desired maximum motion ranges. These ranges can be used to limit program ranges in various ways, for example by controlling computer pointer movements, volume, or other aspects of the system.

The system is adapted to allow multiple people to interact at once, either ‘live’ with all users at the same location, or remotely with some or many users participating from different locations by means of network connections. Examples of multi-person interactions include performing musical pieces with multiple parts and/or instruments, allowing some participants to conduct and others to play, having some write lyrics and others sing, and so on. Forms of competitions and/or collaboration are also possible, for instance a ‘dance-dance-revolution’ type game may be implemented that is useful for building motor coordination and rhythm.

It is possible to control not only the sound coming through loudspeakers and/or headphones by means of the system; further aspects of a room ambience may also be controllable by the system. For instance, the lights and lighting pattern, as well as other physical devices such as drum sets, piano keyboards and the like may be controlled by the system.

To make the system as accessible as possible to as many users as possible, nearly all parameters of the system are controllable and modifiable, including the size of keys shown on screen, size of icons, ranges of motion expected for performing a certain aspect of control, and so on.

It is withing a professional musical ensemble in which there is an ability to adapt to each participant according to physical disability and cognitive learning ability to be part of a professional musical ensemble.—For example, to go on stage with a famous artist, etc., the composition is combined with musicians with normal physical instruments

### Games

A number of different games may be implemented using the inventive system. For example, a shooting game will be familiar to many kids who have grown up with the ‘first person shooter’ genre. The player may for instance shoot different instruments to hear them. In another game, the different players must ‘play’ different instruments by causing their game characters to contact icons representing various instruments, for instance in time with a given piece of music. Points may be disbursed based on any manner of criteria such as musicality, tempo, number of notes played, etc.

Since face recognition is part of the system, games may be controlled by a user's facial expressions. For example, a game action may be controlled by smile, or likewise by speech, movement, or other actions.

In the games as in the rest of the system, one may adjust of almost all parameters in the game according to the physical and cognitive ability of a patient-such as the rate of movement of the objects, the intensity of the sounds, and so on. Thus one may for example make finding hidden objects easy or difficult way. Additionally one can choose any image from the Internet and use it as an object in the game.

### Controls

Controls of the system allow for users to change characteristics of characters, instruments and pointing devices. For instance their size may be changed, characters and instruments may be moved, and so on. Other elements of the system may also be controlled such as an equalizer, volume and pitch control, lighting, and so on.

In contexts in which there are both a user and an operator, the system is designed such that it is easy for both user and operator to make changes to the system, control the particular application being used, change samples, and so on.

The system is designed to be highly customizable. In this vein, it is within provision of the invention to allow the user to download images from the network (for instance by means of an image search) and use these images for avatars or icons.

### Instruments

Both virtual and actual instruments of all forms may be used with the inventive system. Playing the instruments may be based on various characteristics of user movement and other action including characteristics associated with walking, running, moving one's head, pinching movements and other hand movements and gestures in general, speech, movements to or from given points in space, and timing characteristics including speed of movement, synchronization with computer-given cues, and so on.

For example, a user may be tasked with beating a virtual drum in time with a piece of music being played by the computer. The user may change the tempo of song and instruments being played over the music by means of beating the drum at a faster pace, with the input to the computer being accomplished by means of video or other sensors (including structured light, lidar, ultrasound, and any other sensors suitable for purposes of HCl.

It is within provision of the invention that users may create self-defined instruments. For instance, by means of gesture the user may define a number of locations. These locations may then be used to control an instrument. For instance, a virtual flute instrument may be controlled by a set of eight locations, each location corresponding to a note of the scale.

In the same way, a user may define movements or gestures, and assign these movements or gestures to actions required for control of various instruments.

It is within provision of the invention that ‘real life’ non-virtual instruments be controlled by a user interacting with a computer, by allowing for control over these instruments through use of motors and other actuators. These motors and other actuators and associated circuitry may be directly controlled by various outputs of the computer being used for the system, including wireless means such as WIFI, Bluetooth, and the like.

It is envisioned that the inventive system may be found useful for practice of yoga, dance, exercises for the elderly, music-based exercise such as jazzercise, and so on.

It is within provision of the invention to encourage the user to synchronize or separate the motion of various body parts. For example, after experiencing a stroke it is common in certain cases for both hands to be used in parallel without independent control of each hand. In order to attempt to reverse this tendency and restore independent hand control, the system may for instance require the user to control a pointing device using the left hand for horizontal pointer movement, and right hand for vertical pointer movement.

As a further example of the rehabilitative aspects of the invention, for cases of palsy or stroke the user input may be defined as based on control of the tongue, facial expression, or other facial characteristics.

As mentioned a system operator may be involved in use of the system, for instance to endure that initial setup runs smoothly. The operator may control a set of parameters, for instance defining a ‘hover period’-how long the user-controlled pointing device must stay upon an object before it the object is activated in the manner of a mouse click.

It is within provision of the invention that the user may define gestures that are relative to their body position and orientation.

As an example of one type of interaction possible with the system, the user may control notes by stepping in place or while walking. Every step detected by software of the invention causes the computer to play a note. Other gestures defined may for instance include raising the hand to stop the music sample, and lowering the hand to start the music after stopping.

Aspects of the system that may be controlled by the user include tempo, rhythm, and speed; pitch, timbre, tone; and any other aspect of musical production.

The system may use a calibration to fine-tune a voice-control method for HCl, including defining minimum and maximum volume ranges for input and output, speed of speech and pitch thereof, and so on. As part of the musical creative freedom that the system allows, a user or operator can control every aspect of playing and producing music, for example

It is within provision of the invention to employ speech to music methods for users alone or in groups. These methods are adapted to taken spoken words and create music of various sorts, for instance by employing voice control (whereby the user can use voice commands like ‘change instrument’, ‘higher pitch’, ‘lower volume’, ‘faster beat’ and so on, or by means of ‘autotune’ methods that create quantized pitches from the varying pitch of speech, and so on. The speech to music module of the invention is adapted to deal with different languages such that users from around the world may utilize the system without any further localizations required. The speech-to-music provisions of the invention are in addition to the voice control that users may employ with the system for control of all aspects of system operation.

It is within provision of the invention to allow users to define, for instance, which word, sentence, or vocal noise controls which instrument.

Due to the voice input capabilities of the system it is adapted for learning languages. For this purpose, input and output languages may be defined such that input in a given input language is converted by means of speech-to-text algorithms to a given output language and either spoken (using text to speech algorithms), printed onscreen, or both. Icons and/or other images, as well as music suitable to the word or sentence in question may also be displayed for further clarity and to aid the memory.

One particular application of the inventive system allows users to draw onscreen, either singly or in groups. The drawing may be accomplished by (for instance) coloring the screen on positions where the pointing device travels. This can be done while music is playing, with gestures for instance being performed according to the rhythm of the music being played.

### Hardware and Input Devices

Hardware associated with the invention include various HCl devices, as well as speakers, microphones, amplification equipment, and other hardware associated with music and sound production, and devices facilitating use of computers and monitors by those with limited mobility and other impairments. As one example of such hardware, a rigid arc provided with a set of multiply-jointed telescopic holders has been found to be useful for holding various parts of the system (such as screen, keyboard, stylus, input devices such as the Kinect®, and other hardware. In this way the system can be adjusted for use with users of any height and many different physical limitations, including children, the elderly, and differently-abled users of many types.

For users with limitations of bodily movement, eye tracking can be used as a form of input. All aspects of system control can be eye activated, including control of system parameters, such that (for instance) a quadriplegic user may interact with the system to produce music and enjoy therapeutic benefits of music therapy and in some cases even physiotherapy.

As a specific example, use of an eye tracking system for input can be exploited to allow users to (for instance) play notes from a certain scale and paint at same time, all by use of eyes alone. One can paint according to the rhythm of the music or as an expression of the music being played. In addition, the user can control musical parameters at the same time as painting-controlling the volume of notes, speed or tempo, etc. Playing and painting in a group can also be done in real time, where everyone sees what the other is doing. Users can initiate virtual paintbrush color changes, changes to the brush size, and so on.

A music room may be implemented using one or more systems of the type described. This room may be beneficially outfitted with speakers, projectors, and computer-controlled lighting to allow users an immersive light-and-sound experience while controlling the various pieces of software of the invention. The grouping of these parts and the system's ability to adapt to different situations and special hardware products (which sometimes requires customization) allow the system to be the most accessible known to us.

An operator can control which user interfaces are to be employed for a given user, for instance whether the user uses a touch screen, or uses a conventional mouse, or uses a motion tracking device. Furthermore the interaction type may be defined by the operator, for instance as to whether actions occur on ‘hover’ (when the pointer hovers over a given icon for a minimum time) or on ‘click’ (when the user performs an action to be interpreted by the system as a mouse click). The operator of the system can create customized tools for a given user that implements a system for fully and professionally learning music, for control by means of multiple body parts (voice, facial expressions, head movements, hand movements).

Other input devices and sensors including physiological monitors such as heart rate monitors, oxygen perfusion, blood pressure monitors and so on. The output of these monitors may be used to control various aspects of the music being played, such as tempo and speed, intensity, volume, equalization and so on. By these means a form of biofeedback may be achieved, allowing users to control their own heart rates for instance. This may be found of use for those with issues of anxiety or simply for relaxation and meditation. The system can be mobile, implemented at a single position, as a full room for positions in separate use and playing in an ensemble. An operator can control the entire system from a central position and a dedicated tablet through a dedicated application.

Augmented- and virtual-reality hardware may be used with the invention, allowing users to participate in online virtual worlds or to use virtual overlays upon their external reality.

Other hardware devices such as EEG, EMG, TENS, breath detection, gyroscopes, magentometers, accelerometers, and IMUs incorporating these may all be employed with the system as HCl devices, to measure user behavior, and to allow users to interact with the system.

### Music Therapy

It is an object of the invention to facilitate music therapy and in particular music therapy with differently-abled users having various physical handicaps or limitations. One of the goals of the invention is to enable playing and creating for differently-abled users with different disabilities or physical limitations, thus strengthening their emotional and mental state and motivation, and helping them in the progress of treatment/rehabilitation.

As one example of an implementation of the system, a hand tracker may be used to track the hands of (for instance) a user without use of his/her legs and limited mobility in his/her hands. The hand tracker is adapted to track some number of points on the hand which may then be translated key presses for operation of a virtual piano. Furthermore, the user may ‘press’ several points in the air along a path to create a series of notes. As mentioned above, in a calibration step the user can map their own possibly limited range of motion or motions to a different range of motion or motions in a computer representation such as a GUI pointer movement, or in this case individual finger movements of a piano player. Thus, a user who can only move their hands (for example) a few centimeters in either direction can map this movement onto a much larger travel such as an octave range on the piano, allowing them to play full piano pieces. This will be tremendously satisfying for example to pianists who have suffered strokes or other debilitations that impede their range of movement. Augmented reality may also be integrated here as well as virtual reality. For example, combining a virtual piano with real piano keyboard in group work, playing in an ensemble at work with body gestures, and so on. Use of AR and VR goggles of various types may be found of use in this regard. The system can also include a device that sits on parts of the body such as a bracelet or in another way, and can interface with it, and the detection of movement is done using various sensors included in it, unlike cameras, it is a physical device that sits on a patient, for example on the arm or leg without cables, and detects the movements—and can also Write down the movements unique to the patient and work with them. the system can also include a device that sits on parts of the body such as a bracelet or in another way, and can interface with it, and the detection of movement is done using various sensors included in it. Unlike a camera, this is a physical device that sits on a patient, for example on the arm or leg without cables, and detects the movements of the user—and can also record the movements unique to the patient for subsequent work with them.

Another application allows for musical pieces to be played note by note, with the user controlling the tempo or beat. In this case the computer provides the correct sequence of notes while the user controls the rate at which they are played, for instance making a certain movement to progress to the next quarter note or eighth note. In another implementation a system operator plays notes while the patient activates them, allowing the patient to feel like he or she is playing music when in fact he/she is simply activating the next note or chord.

### Physical Therapy

An important aspect of the invention is provision of means and methods of physical therapy, especially in the vein of methods using music in conjunction with physical therapy for users of possibly limited mobility or cognition.

To this end a number of novel instruments and techniques have been developed. For instance, gesture control (e.g. based on the last N seconds of a user's gestures) may be used to control the tempo of a piece of music being played by a computer of the system, or to control the pitch, timbre, or instrument being played by a computer of the system.

Gestures may be physiotherapeutic, and in some cases designed specifically for a particular user. For instance for users requiring therapy of their hands, gestures such as pinching, holding, grabbing and the like may be employed. Likewise, exercises developed for strengthening, improving motor control and coordination, limb independence, and so on can all be utilized with particular implementations of the invention.

### Dance

It is within provision of the invention to include dance and dance therapy within the suite of activities possible using the system. For instance, one application allows users to control different instruments by using different movements, each movement for instance defined beforehand as being associated with a different movement.

Small pieces of music, notes, or samples may be activated by particular user movements, allowing for interactive combined dance and music pieces to be performed, either in choreographed or improvised fashion.

### Music Education

Music education is also possible with the inventive system. For instance the system may be used to learn notes and chords by presenting a representation of the current note or chord being played (by any of the means described above). Alternatively, a game wherein the user must input the next note in a known sequence may be implemented, with the note for instance being chosen amongst a keyboard of possibilities represented onscreen.

Chords may be learned by similar means; each chord for instance may be represented by a box, which can be activated by the user pressing the box using various forms of input. The boxes may be moved by the user for easier operation. Another form of game-based learning uses a ‘Guitar hero’ style operation wherein a set of notes is shown moving from the horizon towards the user, the notes representing a certain piece of music being played. If the user hits the notes correctly they advance in the game. In some embodiments, no prior knowledge of music is needed to operate the system and learn basic music theory and/or performance.

Likewise it is possible to present a class or course allowing for students to learn music in any of the methods outlined above or through more conventional classroom instruction.

### Use Cases

The invention may be found useful in a number of settings including hospitals, clinics, elder care facilities, physiotherapy facilities, special and regular education facilities, music rooms, dance studios, children's play facilities, language learning institutes, music theory and performance facilities, and the like. As mentioned the system may in many cases be used remotely as well, so a setup at (for instance) a music performance institute may be used by a number of remote participants, either singly or in groups.

One therapeutic use case employs music that fits the users mood, which can be useful to allow certain users to express emotions they might not otherwise easily express. For instance, anger, sadness, happiness, anxiety and so on are all expressed in various forms and pieces of music, and by allowing users to create and interact with music of such forms the users may find outlet for expression of such emotion that may otherwise be difficult or impossible to express.

A dietary use case involves for instance use of hardware such as the ‘Makey Makey’ which allows use of most objects as input devices. Thus for instance the invention may employ a set of vegetables as input devices to play game using icons of vegetables.

A ‘group music therapy’ use case has many people playing music together collaboratively, using the inventive system. For instance, each person may be responsible for a particular instrument or section of music. This interaction requires communication between players, which may provide useful training/therapy for users lacking verbal communication skills (for instance), or users not sharing a common language, autistic users, and so on.

It is possible to integrate all the parts of the system, the movements, the gestures, etc. for purposes of playing in an ensemble. For example a group may all accompany an existing or pre-recorded song, or the group may play in a professional ensemble. A singer performing on a stage with professional musicians can perform in combination with patients using the system, empowering the therapeutic population who are generally thrilled to perform before an audience.

### Musical Matching

Various gestures can be translated for any musical need in this system. For examples if a patient moves to a different position than he started from, the system will continue to recognize the selected body part, implementing compensation for position change.

The invention allows for advanced triggers for creating music, including for example: distance between body parts, integration between different body parts, and gestures in general with reference to different fixed points in space. The system is able to record and learn movements in real time. For example one may define certain movements in dance or physical therapy.

### Monitoring Progress

The system allows the user to play and create music without prior knowledge. In addition, it is possible to use the system for learning to play according to different levels of difficulty, customized to the user according to his needs and progress. For example, different ages, cognitive abilities, and motivations for learning/playing experiential music require programs of different levels of cognitive and motor ability. The system can be used both for musical learning, and for the need of a musical/playing experience without much effort. Everything is modular and can even be changed at the operator level.

### Mirroring

In some applications of the inventive system, a ‘mirror character’ is presented. This is a healthy character who does what the user does (in terms of physical movement) and then gives an empowering experience. For example, for facial expressions and skeletal or hand movements, seeing a character on the computer representing a healthy person doing these same movements, the patient experiences him or herself as a healthy person, which can serve as an empowering experience. As another example for a physically limited patient, a combination of the face of the patient for example on the body of a healthy person may likewise serve to give the patient the feeling of mobility.

### OTHER EMBODIMENTS

An example of technical work using an instrument is given here. A user can play an instrument using a set of points in space. For instance, an Azure Kinect sensor can be used to for identification of joint and body movements. By writing scripts through the Unity engine, a graphical display of a user can now be made to appear mirroring the movements of the user. Commands or indicators can be used that identify the specific person playing, such as using an image of the users face on the displayed image, using a name tag or text field on the image, and so on. The joint and movement information may now e sent to a subsequent piece of software such as Max Live, which uses a script with an advanced AI training system that has been written. This script allows, among other things, the user to select a body part and a set of angles for movement (for example, hand movement up and to the side). The user or operator may now use the system to record the positions that the patient chooses in space, and then at the selected positions in space the user can activate different musical options through Ableton Live software, for example, to activate a song that the patient has chosen. This song may actually be played by the patient, in various degrees—for instance the patient may simply advance the song in time by his movements, or may actually change the pitch or volume of his virtual instrument in time to the song. The patient may play a solo of a musical instrument on the section in the song as soon as he is in the position at which this musical instrument was defined. From Ableton Live, the locations that were recorded in relation to their location in space are sent back to Unity, and appear onscreen.

It is also possible to indirectly incorporate points on the patient, for example in the case of people who are difficult to identify on camera automatically. This may be accomplished using object recognition to identify injured people, who hold specific pre-planned objects for this purpose. For example, a disposable cup/=or mug held by a patient may be used. Object recognition software has been prepared for the task of identifying and tracking the cup or other object, and by tracking the object the user movement may be inferred. This will be found useful for people whose skeletal structure is difficult to identify due to deformation from birth defects or physical injury.

Another example involves a physical motion sensor with an adjustable rubber band that can interface with any part of the body, this providing another way to identify any movement of people with severe disabilities who are difficult to identify. Another example would be a lightweight sensor whose movement activates it, for people with severe physical disabilities who, by supporting their minimal body movement, can use this to activate all elements of the system.

### Case Studies

### Case Study: 1 Nutritional Rehabilitation—

In this case, we consider a paraplegic patient one a year after a car accident. The patient did not eat vegetables at all and is repelled by them. The treatment goal was to get the patient to eat at least one or two vegetables a day. The treatment method involved gradual exposure to vegetables.

The inventive system comes into play by implementing a process of gradual exposure to and contact with vegetables. During sessions, the patient was exposed to virtual objects related to vegetables in an interface using music, activating the movement of a physical vegetable while playing a musical tune. This continues until the patient closes a circuit when the body touches an object (in this case an actual vegetable). This touch triggers certain notes and musical rhythms that the patient perceives as a reward.

During the treatment, the patient was observed eating the real “musical” vegetables. This did not happen in other treatment settings such as physiotherapy/occupational therapy, etc. In this way the goal of the treatment was achieved relatively quickly.

### Case Study 2: Speech Therapy

This case involves a patient after a head injury several months prior to treatment. The patient does not communicate at all using normal speech; his attempts at speech are so weak that one cannot hear a sound.

The treatment goal was for the patient to communicate at a medium-high volume in normal speech in order to interact with his environment.

Use of the inventive system involved working in stages, employing a process customized for the patient's rehabilitation:
Stage A: Identification of basic sound production and introduction of a musical trigger.
Stage B: Trigger music by speech volume, then by volume continuity.
Stage C: Trigger on system identification of a word spoken by the patient; on identification of a spoken correct word from a list; and on increasing the number of words per trigger up to a sentence.

A final stage involved combining all these stages as a condition for receiving triggers.

Technological tools for this implementation of the inventive system involved signal processing of audio data, using a microphone for audio input. This system allows for increasing the patient's motivation to increase volume for positive feedback, as well as regulating the positive feedback through technological means and thus gradually encouraging the patient to reach higher volumes and achieving the treatment goals.

The patient completed a series of treatments through which he reached a state where his voice could b heard at a medium-low volume, something which was not possible before.

### Case Study 3: Physiotherapy

This case involved a quadriplegic cerebral palsy (CP) patient, injured on the right side. The treatment goal was to get the patient to make himself coffee with minimal help of a therapist, and to wash his head using two hands.

Treatment methods for this case involved increasing the range of motion of the shoulder and elbow; strengthening the upper limbs in terms of flexion and abduction; and improving balance in static and dynamic standing.

Use the system for this case involved several approaches. For increasing the range of motion of the shoulder and elbow, reaching a desired trigger based on patient movement activates a game action that is perceived as a reward.

For upper limb strengthening, flexion and extension, the following embodiment of the inventive system was used: calibrating the patient's existing range and measuring the increase in the range that occurs during the creation/writing of words, and ‘playing’ the words by dividing the existing range of motion into 7 equal parts to represent 7 written characters of a word.

For improving balance in static and dynamic standing, the following embodiment of the invention was used: Triggers were located at different distances and angles from the patient's position such that the patient has to reach outside his support angle. The system was adjusted using machine learning to the patient's needs and progress, setting goals that are updated visually. Furthermore the location of the triggers in space changes according to the patient's progress in balancing, with triggers being used to activate a drum system that is observed through VR glasses.

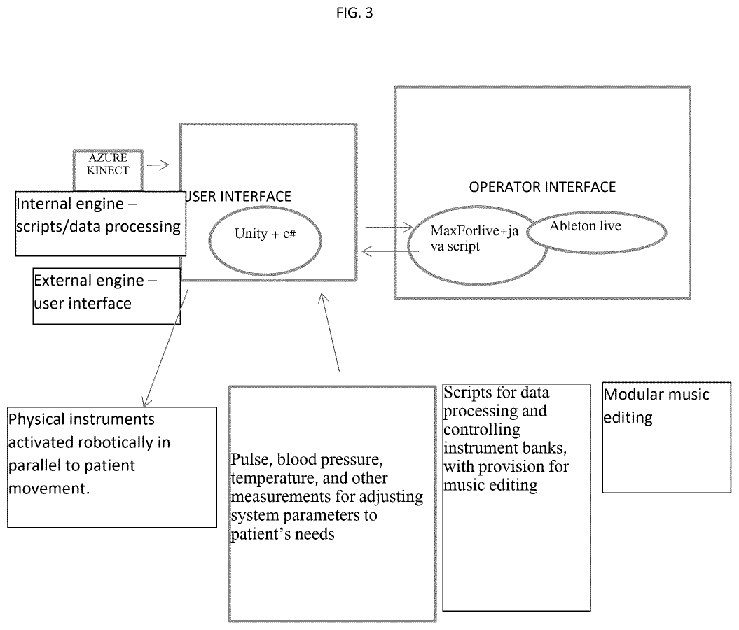

One implementation of the system is shown in FIG. 3. Here we see one implementation of the inventive system.

A list of add-ons in the software program maxForLive using JavaScript scripts for example includes:
Choosing the processing technique: automatic/manual calibration. Point/movement registration system. Mouse cursor movement interface system
Correcting the patient's movement position.
A measurement system that allows measuring various parameters, improving range of motion. Numbering movements above a required height, etc. Treatment history interface of a measurement system and its progress parameters
Various tools for creating music in a modular manner, according to the content, and allowing creating music in a technologically innovative manner without prior knowledge

A list of add-ons for example using Unity and C#scripts includes:

AI identification of the desired user from the existing users in the space. And opening an existing therapeutic array recommended for him and system communication with him

Adjusting the required visual display: Therapeutic visual display of a healthy person's figure\camera reversal\body structure display\marking the active/most active body part in color. Visual display of musical instruments that interfaces with patient movement and music. And more

Real-Time Distance in Cm Between Relevant Body Part and Object with Arrow

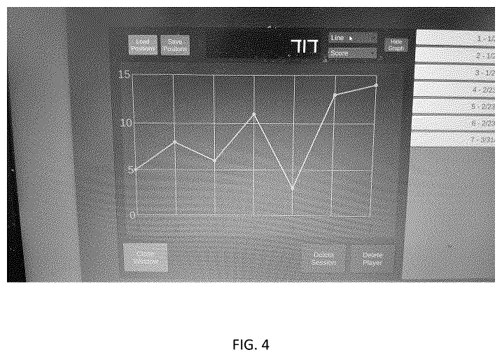

FIG. 4 presents a graph of a user range-of-motion parameter as it changes over time. From the graph one sees a clear improvement (increase) in the range of motion. By means of graphs such as these the patient's progress can be quantified, and this progress may in fact be used as feedback within the system, for example to choose between several alternative treatment methods, with the most effective one or ones (as judged by graphs such as these) being used.

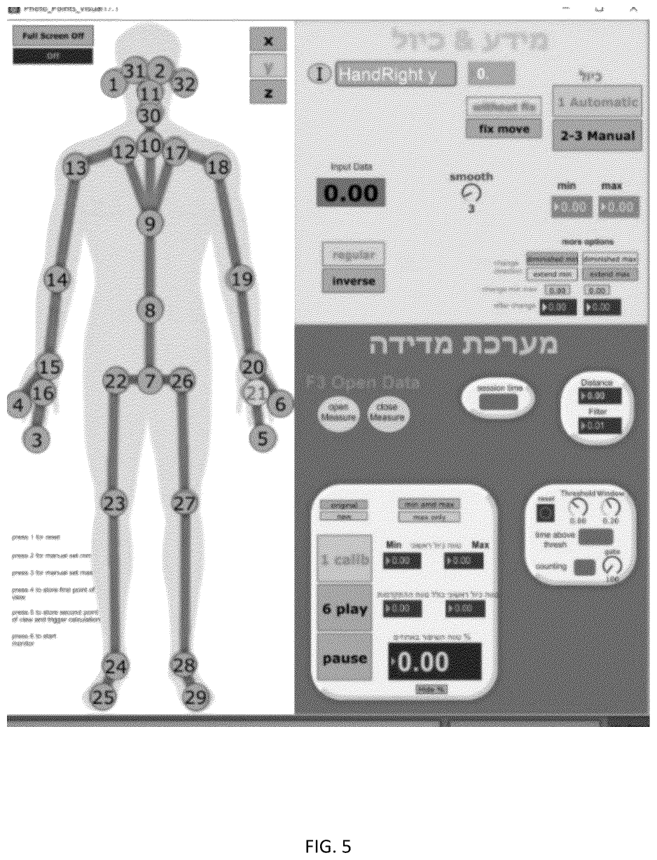

FIG. 5 presents one control screen of the inventive system, with points representing limbs and joints of the body on the left, and calibration and control parameters on the right.

Facial recognition or expression detection can be used to control therapy scenarios. This can be done generally using facial movements at different levels of sensitivity for treatment, rehabilitation for various speech therapy needs and language, etc.

Multiple users can use the system at the same time. This can be done for instance using virtual musical instrument with different shapes, either shared (with each screen showing the same instrument), or split (with each screen showing a different instrument). A musical composition can be combined with other content allowing a group composition or ‘jam’ having measurable rehabilitation goals. This type of scenario also allows the ability to professionally integrate with musicians of different musical instruments, including people with different abilities using real or virtual instruments customized for their particular abilities. The ability to make shared content using computers (possibly including augmented reality), allows for a synchronized and uniform musical experience between people with very different physical and cognitive abilities.

User defined gestures may be created using various algorithms including machine learning. The use of machine learning allows for various advanced functions such as: recording movement/points, averaging points, or repeating a point on the sequence. Furthermore this opens possibilities for automatic calibration, isolation of a specific patient organ using mathematical calculation, and isolating movement (e.g. finger movement alone).

Musical triggers may be modified in terms of level of difficulty according to the user's limitations, with the possibility of gradually increasing difficulty levels according to the pace of treatment.

Personalized libraries can allow for treatments according to population segment, demographics, language, race and so on. This type of information can be used in conjunction with the sensors and other elements of the invention to better adapt to a particular patient's needs.

The system may be used for general visual illustration, for example from using avatars according to therapeutic need, to an inversion display, to a musical instrument display customized to the patient or content that serves its therapeutic purpose musically.

Examples of inputs that can be with the system include hand position or movement for volume of music, a smile for the sound of water or playing a musical role, eyebrows for drums, finger movement down type of drum, hand in a certain position or movement plays a scale, etc.

The invention allows a system of measurements and the ability to monitor information and the progress of such a process systematically in graphs, and other output. This allows users to expand their range of movement, change movement associations, amount of distance a limb can move, amount of time spent in a given location, movement speed graph, and so on.

The invention allows a tool for musical accessibility for rehabilitation purposes and according to personal goals. A combination of areas such as gait, then for example people with cognitive limitations experience an experience of pleasure and success, adjusting the level of difficulty of the musical activity to the therapeutic purpose, etc.

The invention introduces a systematic system for personal and rehabilitation therapeutic purposes with proven experience, a case of success and an advanced organized method in the context of music and other professions. Emotional treatments can be implemented with the system, changing the characteristics of the system as needed.

The invention has mechanisms for working with skeletal processing and movement detection in simple but advanced way. The system allows for automatic calibration, position correction and identification of a limb in isolation despite a change in position, numbering and selecting a person in the camera, advanced AI point system, movement recording, and so on.

The system allows for experiential connection to a multidisciplinary system: painting and playing, playing music and gaming with the system (e.g. playing Snake, shooting games, car games), playing video while painting pictures, etc.

1. A method for facilitating paramedical rehabilitation towards a therapeutic goal for a user comprising steps of:
a. providing a computer with HCl hardware adapted for gathering input from said user;
b. gathering input from said user to define a set of gestures;
c. using said gestures to control aspects of music production by software running on said computer;
d. requiring said user to broaden the range of motion required to produce said gestures, as determined by a paramedical therapist;
whereby to which the production of music is controlled by the user performing gestures which in turn serve to progress towards said therapeutic goal.

2. A method adapted to enable progress in rehabilitative processes and treatments through creating and playing music using a variety of HCl input methods to allow users of various abilities to interact with a music creation system.

3. A system for music creation and physical rehabilitation comprising:
a. one or more HCl input devices including gesture, eye-tracking, and voice input modules;
b. a processor configured to translate said input into real-time musical output;
c. a calibration module that allows automatic input mapping and user definitions for precise personalization according to patient needs;
d. a feedback module providing visual and auditory cues;
wherein the system enables personalized, adaptive music-based therapy.

4. The method of claim 2, wherein user progress is stored over time and visualized using interactive graphs showing improvements in range of motion, speed, static maintenance of movement, walking distance, clicks, volume of speech, accuracy, or cognitive engagement.

5. The method of claim 2, further comprising use of facial recognition or expression detection to control therapy scenarios.

6. The method of claim 2, wherein musical collaboration is supported among multiple users in real time via a networked environment.

7. The system of claim 3, further comprising means for calibrating a user's limited mobility range for use in a musical interface, including means for:
a. automatically or manually detecting physical motion of a body part;
b. automatically or manually mapping said motion to a range of virtual instrument control;
c. progressively increasing target motion thresholds;
whereby therapeutic motion targets are integrated into musical performance tasks.

8. The system of claim 3, wherein the gesture recognition includes user-defined gestures captured through depth-sensing cameras and machine learning algorithms.

9. The system of claim 3, wherein said eye-tracking module allows users to trigger musical notes or instrument parameters based on gaze position, duration, or movement direction.

10. The system of claim 3, further comprising a voice input module configured to detect vocal parameters including pitch, amplitude, and phoneme clarity to control system outputs and provide speech modification and feedback.

11. The system of claim 3, further comprising real-time monitoring of user physiological data including heart rate, respiration, and muscle tension to modulate musical parameters as biofeedback.

12. The system of claim 3, wherein virtual and physical instruments are triggered by user movement through actuators connected to the control system.

13. The system of claim 3, wherein said software includes integration with commercial music production environments.

14. The system of claim 3, further comprising a library of therapeutic programs tailored for distinct user populations, including children with autism, elderly users, and stroke survivors.

15. The system of claim 3, wherein the HCl interface allows configuration by a therapist to set treatment goals, assign motion-to-music mappings, and monitor user performance data.

16. The system of claim 3, further comprising a drawing module enabling users to paint or sketch onscreen using gaze or motion input synchronized with music playback.

17. The system of claim 3, further comprising customizable virtual avatars or mirror characters that reflect or amplify the user's actions to encourage emotional engagement and perceived self-efficacy.

18. The system of claim 3, further comprising a mode for triggering predefined musical sequences upon detection of movement patterns involving multiple body parts in synchrony.

19. The method of claim 4, further comprising the step of associating specific gestures with musical notes, chords, or rhythm patterns.

20. The method of claim 4, wherein said mapping includes nonlinear scaling between physical and virtual ranges to accommodate differential control precision across motion extents.
